(19) United States
(12) Patent Application Publication
(10) Pub. No.: US 2013/0164352 A1
(43) Pub. Date: Jun. 27, 2013

(54) ANTIPERSPIRANT COMPOSITIONS AND PRODUCTS EFFECTIVE TO FACILITATE PREVENTING, REMOVING OR MINIMIZING FABRIC STAINS AND METHODS FOR MAKING THE SAME

(71) Applicants: Travis T. Yarlagadda, Phoenix, AZ (US); Timothy J. Taylor, Phoenix, AZ (US); Tasha Desai, Phoenix, AZ (US)

(72) Inventors: Travis T. Yarlagadda, Phoenix, AZ (US); Timothy J. Taylor, Phoenix, AZ (US); Tasha Desai, Phoenix, AZ (US)

(73) Assignee: The Dial Corporation, Scottsdale, AZ (US)

(21) Appl. No.: 13/334,407

(22) Filed: Dec. 22, 2011

(51) Int. Cl. A61K 8/02; A61Q 15/00

(52) U.S. Cl. 424/401; 424/65

(57) ABSTRACT

Antiperspirant compositions, products, and methods for making antiperspirant compositions and products are provided herein. In an exemplary embodiment, an antiperspirant composition comprises an active antiperspirant compound and an active cleaning agent that is effective to facilitate preventing, removing or minimizing fabric stains.

### TECHNICAL FIELD

The present invention relates generally to antiperspirant compositions and products, and methods for making the same, and more particularly relates to antiperspirant compositions that are effective to facilitate preventing, removing or minimizing fabric stains such as on garments worn by antiperspirant users, antiperspirant products comprising such antiperspirant compositions, and methods for making such antiperspirant compositions and products.

### BACKGROUND

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

Stick antiperspirant products include an antiperspirant composition within a container. During use of the stick antiperspirant product, the top of the container is removed and the application surface of the composition is contacted with the skin, such as the underarm, by swiping or rubbing the stick across the skin. Sometimes the product also includes an undercap, or factory seal, that covers the application surface and is removed prior to first use. The container generally also includes some mechanism for moving the composition upwards through the container to continue to provide an exposed application surface.

Fabric staining on garments, e.g., especially in an underarm area, worn by antiperspirant users has long been a concern with antiperspirant use. There are various factors that are believed to cause fabric staining. First, the acidic nature of the active antiperspirant compound in combination with perspiration can cause fabric degradation to occur over time due to repeated and prolonged exposure. A second factor is the presence of iron in the antiperspirant composition, such as in the active antiperspirant compound(s), clay(s), and/or fragrance(s), which can transfer to the garment and oxidize or enhance oxidation of the active ingredients. Another factor is the presence of iron, calcium, and/or other inorganic metals found in the wash water that is used to wash a soiled garment previously worn by the antiperspirant user. These inorganic metals can inhibit thorough removal of the antiperspirant ingredients resulting in a buildup of antiperspirant on the garment after multiple wearings and washings, and/or the inorganic metals can precipitate onto the garment to cause fabric staining. Efforts to address fabric staining typically include incorporating a less acidic active antiperspirant compound(s) into the antiperspirant composition. However, many of these reduced active antiperspirant compounds have less antiperspirant efficacy than higher composition active antiperspirant compounds. Also, fabric staining caused by factors other than the acidic nature of the active antiperspirant compound, e.g., presence of iron in the antiperspirant composition, inorganic metals present in the wash water, antiperspirant buildup on the garment, and/or the like, are not addressed by simply using an antiperspirant composition with a less acidic active antiperspirant compound(s).

Accordingly, it is desirable to provide antiperspirant products that exhibit strong antiperspirant efficacy and that address fabric staining of garments worn by antiperspirant users. Furthermore, other desirable features and characteristics of the present invention will become apparent from the subsequent detailed description of the invention and the appended claims, taken in conjunction with the accompanying drawings and this background.

### BRIEF SUMMARY

Antiperspirant compositions, products, and methods for making antiperspirant compositions and products are provided herein. In an exemplary embodiment, an antiperspirant composition comprises an active antiperspirant compound and an active cleaning agent that is effective to facilitate preventing, removing or minimizing fabric stains.

In accordance with another exemplary embodiment, an antiperspirant product is provided. The antiperspirant product comprises a container. An antiperspirant composition is housed within the container. The antiperspirant composition comprises an active antiperspirant compound and a detergent encapsulate that is effective for preventing, removing or minimizing fabric stains. The detergent encapsulate comprises an active cleaning agent and an encapsulating material.

In accordance with another exemplary embodiment, a method for making an antiperspirant product is provided. The method comprises the steps of mixing antiperspirant ingredients comprising an active antiperspirant compound together to form an antiperspirant premix. A molten wax material, the antiperspirant premix, and a detergent encapsulate that comprises an active cleaning agent and an encapsulating material are mixed together to form an antiperspirant composition. The antiperspirant composition is deposited into a mold and is allowed to solidify.

### BRIEF DESCRIPTION OF THE DRAWINGS

The present invention will hereinafter be described in conjunction with the following drawing figures, wherein like numerals denote like elements, and wherein:

FIG. 1 is a perspective view of an antiperspirant product in accordance with an exemplary embodiment;

FIG. 2 is a top view of the antiperspirant product depicted in FIG. 1;

FIG. 3 is an exploded perspective view of an antiperspirant product in accordance with an exemplary embodiment; and

FIG. 4 is a flowchart of a method for making an antiperspirant product in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding background or the following detailed description.

The various embodiments contemplated herein relate to antiperspirant compositions that exhibit strong antiperspirant efficacy and that are effective to facilitate preventing, removing or minimizing fabric stains, antiperspirant products comprising such antiperspirant compositions, and methods for making such antiperspirant compositions and products. Unlike the prior art, the exemplary embodiments taught herein form an antiperspirant composition that comprises an active antiperspirant compound and an active cleaning agent. The active antiperspirant compound is effective to prevent and/or block the secretion of perspiration and/or its accompanying odors. The active cleaning agent is effective to facilitate preventing, removing or minimizing fabric stains, such as on a garment worn by an antiperspirant user.

In an exemplary embodiment, the antiperspirant composition comprises a detergent encapsulate that comprises an encapsulating material that encapsulates and/or retains the active cleaning agent therein. The encapsulating material can be a matrix or membrane material formed, for example, of starch or modified starch that releases the active cleaning agent upon contact with skin, perspiration, and/or washing water, e.g., water used to wash a soiled garment previously worn by an antiperspirant user. As such, the active cleaning agent is exposed and can work to prevent, remove or minimize a fabric stain on the garment that may have been caused or otherwise caused from antiperspirant use.

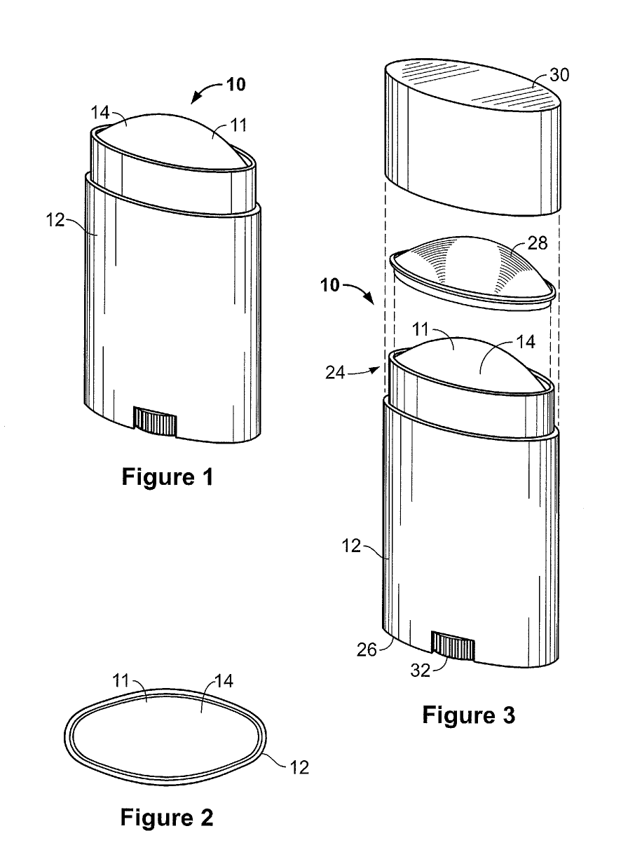

Referring to FIGS. 1 and 2, an antiperspirant product 10 in accordance with an exemplary embodiment is provided. The antiperspirant product 10 comprises an antiperspirant composition 11, such as a solid wax antiperspirant formulation. As illustrated, the antiperspirant composition 11 has an application surface 14 that is substantially dome-shaped and that is configured to be applied to skin, such as, for example, an underarm. The antiperspirant product 10 may also comprise a container or dispenser 12 for dispensing the antiperspirant composition 11 to the skin.

The antiperspirant composition 11 contains at least one active ingredient (i.e. active antiperspirant compound), typically metal salts, that are thought to reduce perspiration by diffusing through the sweat ducts of eccrine glands and apocrine glands and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, such as inorganic and organic salts of aluminum, zirconium, and zinc, as well as mixtures thereof. Some examples are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Some zirconium salt examples are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof.

The active antiperspirant compound is preferably in a perspiration-reducing effective amount. In one embodiment, the antiperspirant composition 11 comprises an active antiperspirant compound present in the amount of from about 5 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycine.

The antiperspirant composition 11 contains an active cleaning agent (e.g. detergent ingredient(s)) that is effective to facilitate preventing, removing or minimizing fabric stains. In an exemplary embodiment, the active cleaning agent is present in an amount of from about 0.05 to about 6 wt. %, such as from about 0.4 to about 3 wt. %, of the antiperspirant composition 11.

In an exemplary embodiment, the active cleaning agent comprises a surfactant, an acidic cleaning component, an alkaline builder, a water conditioner, an antioxidant, a soil release polymer, an oxidizing agent, an enzyme, a corrosion inhibitor, glycol ether, and/or butylcellosolve. The term “surfactant” as used herein in reference to the active cleaning agent refers to a cleaning agent ingredient(s) that lowers the surface tension of water, e.g., perspiration or wash water, so that the water is more likely to interact with soil materials, e.g., material(s) causing or forming a fabric stain, to facilitate preventing, removing or minimizing a fabric stain. The term “acidic cleaning component” refers to a cleaning agent ingredient(s) that lowers the pH of water (e.g. perspiration or wash water) to a pH range that facilitates preventing, removing or minimizing a fabric stain. The term “alkaline builder” as used herein refers to a cleaning agent ingredient(s) that increases, buffers, and/or stabilizes the pH of water (e.g. perspiration or wash water) to a pH range that facilitates preventing, removing or minimizing a fabric stain. The term “water conditioner” as used herein refers to a cleaning agent ingredient(s), such as a sequestering agent and/or a chelating agent, that neutralizes, combines with, and/or removes iron, calcium, and/or other inorganic metals in water (e.g. perspiration or wash water), and/or helps remove antiperspirant buildup to facilitate preventing, removing or minimizing a fabric stain. The term “antioxidant” as used herein refers to a cleaning agent ingredient(s) that reduces or prevents oxidation of iron and/or other inorganic metals in water and/or antiperspirant to facilitate preventing, removing or minimizing a fabric stain. The term “soil release polymer” as used herein refers to a polymeric cleaning agent ingredient(s) that protects the fibers of a fabric by reducing the affinity of soil materials to cling to a fabric surface to facilitate preventing, removing or minimizing a fabric stain. The term “oxidizing agent” as used herein refers to a cleaning agent ingredient(s) that can oxidize and/or react with soil materials to facilitate preventing, removing or minimizing a fabric stain. The term “enzyme” as used herein refers a microorganism cleaning agent ingredient(s) that facilitates preventing, removing or minimizing a fabric stain. The term “corrosion inhibitor” as used herein refers to a cleaning agent ingredient(s) that prevents corrosion or oxidation of inorganic metals in water (e.g. perspiration or wash water) to facilitate preventing, removing or minimizing a fabric stain.

Some examples of surfactants suitable as cleaning agent ingredients include surfactants having a head group that is anionic, cationic, amphoteric, or nonionic, and a tail group. Examples of ionic head groups include head groups having a negative charge and comprising sulfates, sulfonates, phosphates, carboxylates, and/or the like. Examples of cationic head groups include head groups having a positive charge and comprising amines, quaternary ammonium cation, and/or the like. Examples of amphoteric head groups include head groups having both a positive charge and a negative charge, and comprising sulfonates, carboxylates, phosphates, and/or the like. Examples of nonionic head groups include head groups having no charge and comprising fatty alcohols and/or the like. The term “fatty” as used herein is intended to include hydrocarbon chains of about 8 to 30 carbon atoms, such as about 12 to 18 carbon atoms. Examples of tail groups include tail groups comprising hydrocarbon chain(s), alkyl ether chain(s) such as an ethoxylated or propoxylated chains, fluorocarbon chain(s), siloxane chain(s), and/or the like. In an exemplary embodiment, the cleaning agent comprises fatty alcohol ethoxylate as a surfactant.

Some examples of acidic cleaning components include phosphoric acid, nitric acid, sulfamic acid, sodium acid sulfate, hydrochloric acid, hydroxyacetic acid, citric acid, gluconic acid, formic acid, acetic acid, propanoic acid, and/or the like. Some examples of alkaline builders include sodium hydroxide, potassium hydroxide, tri-sodium phosphate, alkaline builder salts, and/or the like. Examples of alkaline builder salts include sodium, potassium, or ammonium salts of phosphates, silicates, or caronates. Some examples of water conditioners include sequestering agents and/or chelating agents. Examples of sequestering agents include sodium tripolyphosphate, tetra-potassium pyrophosphate, organo-phosphates, polyelectrolytes, and/or the like. Examples of chelating agents include sodium gluconate, ethylene diamine tetracidic acid, and/or the like. Some examples of oxidizing agents include sodium gluconate, ethylene diamine tetracidic acid, and/or the like. Some examples of enzymes include protease, lipase, amylase, mannanase, and/or the like.

In an exemplary embodiment, the active cleaning agent is encapsulated and/or retained in an encapsulating material to form a detergent encapsulate. The encapsulating material can be, for example, a starch based matrix, a modified starch based matrix, a starch based membrane, a modified starch based membrane, a carboxylate based matrix, a carboxylate based membrane, polyvinyl alcohol, and/or sodium starch octenylsuccinate. Other encapsulating materials for encapsulating detergent ingredients known to those skilled in the art may also be used.

In an exemplary embodiment, the detergent encapsulate comprises the encapsulating material present in an amount of from about 1 to about 90 weight percent (wt. %) of the detergent encapsulate. In one embodiment, the detergent encapsulate comprises the active cleaning agent present in an amount of from about 10 to about 99 wt. % of the detergent encapsulate. In an exemplary embodiment, the antiperspirant composition 11 comprises the detergent encapsulate present in an amount of from about 0.1 to about 6 wt. %, such as from about 0.5 to about 3 wt. %, of the antiperspirant composition 11.

The inventors have found that by having the active cleaning agent encapsulated or retained in the encapsulating material, the active cleaning agent can be released from the encapsulating material to work on preventing, removing or minimizing a fabric stain at a predetermined time or event. In one example, the encapsulating material is selected and formed to rupture upon frictional application of the antiperspirant composition 11 to skin to release the active cleaning agent. In another example, the encapsulating material is selected and formed to release (e.g., via rupturing, dissolving, or the like) the active cleaning agent upon exposure to perspiration, or alternatively, upon exposure to wash water that is used to wash a soiled garment previously worn by the antiperspirant user. For instance, the encapsulating material may release the active cleaning agent upon exposure to perspiration and transfer to the user's garment to prevent a fabric stain from developing, or alternatively, transfer to the user's garment to work on an existing fabric stain to remove or minimize the fabric stain.

The antiperspirant composition 11 may comprise an anhydrous, hydrophobic vehicle, which includes a volatile silicone and/or a high melting component. In an exemplary embodiment, the active antiperspirant compound is suspended in the anhydrous, hydrophobic vehicle.

The high melting components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, such as from about 2 to about 15 wt. %, of the antiperspirant composition 11. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant composition 11 contemplated herein also may comprise additives, such as those used in conventional antiperspirants. For example, in addition to antiperspirant efficacy, the antiperspirant composition 11 may comprise additives that cause the antiperspirant composition 11 to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in an amount of from about 0 to about 20 wt. % of the antiperspirant composition 11.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the antiperspirant composition 11 may include up to about 5% fragrance or about 2% colorant by weight.

As noted above, in addition to an active antiperspirant compound, the antiperspirant composition 11 may comprise a component or components that cause it to exhibit or impart a desired function or purpose in addition to antiperspirant efficacy. For example, the antiperspirant composition 11 may comprise deodorant active ingredients. A suitable deodorant active ingredient is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor. “Fragrances” as used herein are not considered deodorant active ingredients. Examples of deodorant actives ingredients include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the antiperspirant composition 11 may comprise fragrances, for example, in an amount that imparts a long-lasting fragrance to the antiperspirant composition 11.

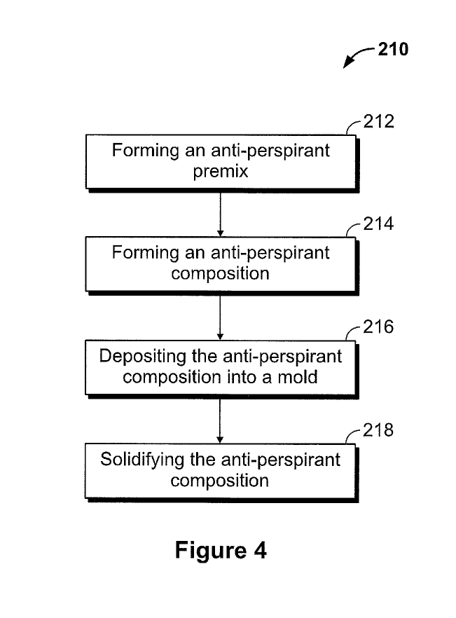

In accordance with exemplary embodiments, a method 210 for making the antiperspirant product 10 illustrated in FIGS. 1 and 2 is shown in FIGS. 3-4. With reference to FIG. 4, the method 210 comprises mixing antiperspirant ingredients including an active antiperspirant compound together to form an antiperspirant premix (step 212). In one example, cyclopentasiloxane is agitated in a mixing container. Fumed silica (e.g. Aerosil R872) and silica (Aerosil 300) are incrementally added to the mixing container and agitated until all of the silica is wetted with the cyclopentasiloxane. An active antiperspirant compound (e.g. aluminum zirconium pentachlorohydrex-GLY) is then slowly added to the mixing container and is mixed until the blend appears homogeneous. High shear mixing is then used to mix the blend until the appearance is consistently fluid and absent of particulates to form the antiperspirant premix.

A molten wax material, the antiperspirant premix, and a detergent encapsulate that comprises an active cleaning agent and an encapsulating material are mixed together to form the antiperspirant composition (step 214) as discussed in the foregoing paragraphs. In one example, castor wax is combined with PPG-14 butyl ether and heated to a temperature of about 85° C. or less and slowly agitated to form a molten wax mixture. The antiperspirant premix and detergent encapsulate are added to the molten wax mixture and are mixed together at a temperature of from about 55 to about 69° C. until the mixture is homogeneous to form the antiperspirant composition (step 214).

Referring to FIGS. 3-4, the antiperspirant composition 11 is deposited in molten form into a mold (step 216) and solidified (step 218). The molten temperature of the antiperspirant composition 11 is generally in the range of from about 50 to about 85° C. The antiperspirant composition 11 is cooled below about 50° C., to produce the antiperspirant product 10 in a solid form. The container 12 may be used as the mold for the antiperspirant product 10 illustrated in FIG. 3. It will be appreciated, however, that the invention is not limited to use of the container 12 as a mold and that any satisfactory mold may be used for manufacturing the antiperspirant product.

In an exemplary embodiment, the container 12 has an application end 24 and an opposite end 26. The container 12 also contains a factory seal 28, which is positioned over the application surface 14 of antiperspirant composition 11 to protect it during shipment and to render it tamper-proof prior to purchase, and a cover 30. The factory seal 28 is removed by the user, and the cover is used during storage of the product between uses. As the product is exhausted, it is advanced from the container 12 by the user using an advancement device 32, e.g., a screw mechanism as shown, at opposite end 26 of container 12.

In an exemplary embodiment, the antiperspirant composition 11 is molded into the container 12 by first sealing the application end 24 of the container 12 with the factory seal 28 and introducing a predetermined quantity of molten antiperspirant composition 11 through the open opposite end 26. The antiperspirant composition 11 is then cooled to its non-molten form for example, by passing the filled container through a forced air tunnel operating at between about 10 to about 25° C. The finished product (FIG. 3) is completed by sealing the open opposite end 26 with a package base (not shown) that includes the advancement device 32. Other suitable methods for molding and/or forming the antiperspirant product known to those skilled in the art may also be used.

The following is an example of an antiperspirant product in accordance with an exemplary embodiment. The example is provided for illustration purposes only and is not meant to limit the various embodiments of the antiperspirant product in any way. All materials are set forth in weight percent.

### EXAMPLE

### Antiperspirant Composition

Antiperspirant Product—Solid Wax Formulation

| Ingredient | Wt. % |
|---|---|
| Cyclopentasiloxane | 32 to 46 |
| Stearyl Alcohol | 15 to 24 |
| Aluminum Zironium | 5 to 25 |
| Pentachlorohydrex | |
| PPG-14 Butyl Ether | 7 to 15 |
| Hydrogenated Castor Oil | 1 to 8 |
| Myristal Myristate | 1 to 8 |
| Silica | 0.1 to 6 |
| Silica Dimethyl Silylate | 0.1 to 6 |
| Perfum | 0.1 to 6 |
| Detergent Encapsulate | 0.1 to 6 |
| Total | 100.0 |

Detergent Encapsulate—Formulation

| Ingredient | Wt. % |
|---|---|
| Fatty Alcohol Ethoxylate C12-14 | 0.01 to 2 |
| Sodium Citrate | 0.1 to 6 |
| Phosphoric Acid | 1 to 8 |
| Citric Acid | 4 to 15 |
| Formic Acid | 0.1 to 6 |
| Sodium Hydroxide | 0.1 to 6 |
| Polydimethyl Siloxane | 0.0 to 1 |
| Zinc Ricinoleate | 0.1 to 6 |
| Encapsulating Material | 50 to 90 |
| Total | 100.0 |

Accordingly, antiperspirant compositions that exhibit strong antiperspirant efficacy and that are effective to facilitate preventing, removing or minimizing fabric stains, antiperspirant products comprising such antiperspirant compositions, and methods for making such antiperspirant compositions and products have been described. Unlike the prior art, the exemplary embodiments taught herein form an antiperspirant composition that comprises an active antiperspirant compound and an active cleaning agent. The active antiperspirant compound is effective to prevent and/or block the secretion of perspiration and/or its accompanying odors. The active cleaning agent is effective to facilitate preventing, removing or minimizing fabric stains, such as, for example, on a garment worn by an antiperspirant user.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. An antiperspirant composition comprising:
a solid wax material having an application surface and comprising, dispersed throughout the solid wax material:
an active antiperspirant compound; and
an active cleaning agent effective to facilitate preventing, removing or minimizing fabric stains.

2. The antiperspirant composition of claim 1, further comprising a detergent encapsulate that comprises the active cleaning agent and an encapsulating material.

3. The antiperspirant composition of claim 2, wherein the encapsulating material comprises a starch based matrix, a modified starch based matrix, a starch based membrane, a modified starch based membrane, a carboxylate based matrix, a carboxylate based membrane, polyvinyl alcohol, sodium starch octenylsuccinate, or combinations thereof.

4. The antiperspirant composition of claim 2, wherein the detergent encapsulate comprises the encapsulating material present in an amount of from about 1 to about 90 wt. % of the detergent encapsulate.

5. The antiperspirant composition of claim 2, wherein the detergent encapsulate comprises the active cleaning agent present in an amount of from about 10 to about 99 wt. % of the detergent encapsulate.

6. The antiperspirant composition of claim 2, wherein the detergent encapsulate is present in an amount of from about 0.1 to about 6 wt. % of the antiperspirant composition.

7. The antiperspirant composition of claim 1, wherein the active cleaning agent is present in an amount of from about 0.05 to about 6 wt. % of the antiperspirant composition.

8. The antiperspirant composition of claim 1, wherein the active cleaning agent comprises a surfactant, an acidic cleaning component, an alkaline builder, a water conditioner, an antioxidant, a soil release polymer, an oxidizing agent, an enzyme, a corrosion inhibitor, glycol ether, butylcellosolve, or combinations thereof.

9. The antiperspirant composition of claim 8, wherein the surfactant comprises sulfates, sulfonates, phosphates, carboxylates, amines, quaternary ammonium cation, fatty alcohols, or combinations thereof.

10. The antiperspirant composition of claim 9, wherein the active cleaning agent comprises fatty alcohol ethoxylate.

11. The antiperspirant composition of claim 8, wherein the acidic cleaning component comprises phosphoric acid, nitric acid, sulfamic acid, sodium acid sulfate, hydrochloric acid, hydroxyacetic acid, citric acid, gluconic acid, formic acid, acetic acid, propanoic acid, or combinations thereof.

12. The antiperspirant composition of claim 8, wherein the alkaline builder comprises sodium hydroxide, potassium hydroxide, tri-sodium phosphate, alkaline builder salts, or combinations thereof, wherein the alkaline builder salts comprise sodium, potassium, or ammonium salts of phosphates, silicates, or caronates.

13. The antiperspirant composition of claim 8, wherein the water conditioner comprises a sequestering agent, a chelating agent, or combinations thereof, wherein the sequestering agent comprises sodium tripolyphosphate, tetra-potassium pyrophosphate, organo-phosphates, polyelectrolytes, or combinations thereof, and wherein the chelating agent comprises sodium gluconate, ethylene diamine tetracidic acid, or combinations thereof.

14. The antiperspirant composition of claim 8, wherein the oxidizing agent comprises hypochlorite, perborate, or combinations thereof.

15. The antiperspirant composition of claim 8, wherein the enzyme comprises protease, lipase, amylase, mannanase, or combinations thereof.

16. An antiperspirant product comprising:
a container; and
an antiperspirant composition housed within the container and comprising:
a solid was material having an application surface and comprising, dispersed throughout the solid wax material,
an active antiperspirant compound; and
a detergent encapsulate that is effective for preventing, removing or minimizing fabric stains and that comprises an active cleaning agent and an encapsulating material.

17. The antiperspirant product of claim 16, wherein the detergent encapsulate is present in an amount of from about 0.1 to about 6 wt. % of the antiperspirant composition.

18. The antiperspirant product of claim 16, wherein the detergent encapsulate is present in an amount of from about 0.5 to about 3 wt. % of the antiperspirant composition.

19. The antiperspirant product of claim 16, wherein the active cleaning agent comprises a surfactant, an acidic cleaning component, an alkaline builder, a water conditioner, an antioxidant, a soil release polymer, an oxidizing agent, an enzyme, a corrosion inhibitor, glycol ether, butylcellosolve, or combinations thereof.

20. A method for making an antiperspirant product, the method comprising the steps of:
mixing antiperspirant ingredients comprising an active antiperspirant compound together to form an antiperspirant premix;
mixing a molten wax material, the antiperspirant premix, and a detergent encapsulate that comprises an active cleaning agent and an encapsulating material together to form an antiperspirant composition;
depositing the antiperspirant composition into a mold; and
allowing the antiperspirant composition to solidify.
